(19) United States
(12) Patent Application Publication
(10) Pub. No.: US 2026/0153504 A1
(43) Pub. Date: Jun. 4, 2026

(54) BIOSENSOR SYSTEM FOR DIAGNOSIS OF FAMILIAL MEDITERRANEAN FEVER(FMF) DISEASE

(71) Applicant: IZMIR BIYOTIP VE GENOM MERKEZI, Izmir (TR)

(72) Inventors: Arif Engin CETIN, Izmir (TR); Idil KARACA ACARI, Malatya (TR); Seda Nur TOPKAYACETIN, Izmir (TR); Burhan ATES, Malatya (TR); Suleyman KOYTEPE, Malatya (TR); Turgay SECKIN, Malatya (TR); Sevket Erbil UNSAL, Izmir (TR); Balahan MAKAY, Izmir (TR); Ceyhun ACARI, Malatya (TR); Ismet YILMAZ, Malatya (TR)

(73) Assignee: IZMIR BIYOTIP VE GENOM MERKEZI, Izmir (TR)

(21) Appl. No.: 19/122,274

(22) PCT Filed: Nov. 22, 2023
(86) PCT No.: PCT/TR2023/051370

§ 371 Date: Apr. 18, 2025

(30) Foreign Application Priority Data
Nov. 23, 2022 (TR) 2022/017709

(51) Int. Cl. G01N 33/543; G01N 21/552; G01N 33/68

(52) U.S. Cl. CPC G01N 33/54373; G01N 21/554; G01N 33/6893; G01N 2201/0221; G01N 2201/023; G01N 2333/46; G01N 2800/24

(57) ABSTRACT

Provided is a portable plasmonic biosensor system to be used in the diagnosis of the Familial Mediterranean Fever (FMF) disease. The portable plasmonic biosensor system includes a substrate, a metal nanoparticle, a ligand protein, a plasmonic chip, at least one light source, at least one spectrometer, and a processing unit.

27

### CROSS REFERENCE TO THE RELATED APPLICATIONS

This application is the national phase entry of International Application No. PCT/TR 2023/051370, filed on Nov. 22, 2023, which is based upon and claims priority to Turkish Patent Application No. 2022/017709, filed on Nov. 23, 2022, the entire contents of which are incorporated herein by reference.

### TECHNICAL FIELD

The invention relates to a portable biosensor system that analyzes the amount of pyrin protein and includes a metal nanoparticle-based plasmonic chip, spectral reading assembly and data processing algorithm for the diagnosis of FMF (Familial Mediterranean Fever) disease.

### BACKGROUND

Familial Mediterranean Fever (FMF) is a rather prevalent autosomal recessive genetic disease worldwide. FMF disease is defined as the impairment of the response of the organism to inflammation as a result of mutations in the MEFV gene. The MEFV gene encodes the pyrin protein. It is mostly found in neutrophils and macrophages and plays a key role in apoptosis and inflammatory pathways. Mutated pyrin causes an unexpected inflammatory response with uncontrolled interleukin-1 secretion (Stella et al. 2019, Giancane et al. 2014, Matzner et al. 2000). Mutations in the MEFV gene cause the synthesis of the pyrin protein, which increases to an abnormal level, and the control of the pyrin protein on inflammation is impaired. Although it is usually diagnosed at a young age, it may not be diagnosed until adulthood in individuals whose attacks are not frequent and severe, and the person may feel completely normal between attacks. Early diagnosis of FMF, which is a common disease, is very important in terms of treating the disease and preventing complications that may occur.

According to national or international patent, utility model and registration searches related to the subject matter of the invention, there is no similar system or product subject to the invention. Patent WO2019048569A1 is based on the measurement of the level of cytokine (IL-18 or IL-1beta) secreted by the immune primer for the detection of FMF disease. This study revealed that FMF patients can be distinguished from healthy individuals by determining the amount of cytokine release or real-time cell death kinetics. The biomarker used in the invention introduced in this document is the cytokine, and although said kit is not a portable platform or a biosensor, it includes the measurement of the cytokine amounts of healthy and sick individuals with classical methods. Therefore, this patent document differs from the said invention in terms of the platform difference, the difference in the biomarker used, the potential to be transformed into a method and product.

There is no examination finding or unique laboratory test that allows for a definitive diagnosis of FMF disease. Since FMF disease is similar to many other diseases in terms of patient complaints and clinical findings, the diagnosis of the disease is quite challenging and delayed. FMF is diagnosed based on clinical findings, family history, biochemical and genetic laboratory data, response to treatment and exclusion of other familial periodic fever syndromes. The results of routine blood tests during acute attacks are non-specific. Today, the definitive diagnosis of FMF is made only by genetic tests that support clinical findings, and there is no alternative diagnostic test to these genetic tests. In addition to the high cost of genetic tests, considering the test results in a long period of 2-3 months and the attacks that patients may have during this period, classical methods used today can cause very serious problems. Therefore, there is a need for a new alternative diagnostic method that will solve the problems related to the diagnosis of FMF disease.

### SUMMARY

Due to the disadvantages of genetic tests, which are the only method for the definitive diagnosis of FMF, and the absence of any other method or device in this diagnostic area, the portable, fast, cost-efficient, and high-accuracy biosensor system developed within the scope of the invention offers a powerful alternative to FMF diagnostic methods.

The advantages of the invention over genetic tests are listed below:
i. Low-cost; The cost of genetic tests is quite high because it includes expenses such as sample preparation, transporting the prepared samples to the center to be sequenced, sequencing and processing the sample data with bioinformatics. Also, sequencing and processing of genetic data requires sophisticated and expensive devices, whereas the invention described in this document is described as a low-cost device.
ii. Faster results; The process of transporting the samples to the center for testing, waiting for a certain queue at this center, performing the tests and converting the test results into meaningful data through bioinformatics analysis can take long periods of time in genetic tests. The invention, on the other hand, shortens the FMF diagnosis process considerably by enabling the tests to be performed directly and at the health center where the patients are resided, given that they are available in every health center due to their low cost.

Given the user-friendliness, portability, and low-cost testing opportunity that the invention will provide, the potential for commercialization is quite high compared to classical FMF diagnostic methods based on disease symptoms and genetic tests.

### BRIEF DESCRIPTION OF THE DRAWINGS

The figures and related descriptions required for the invention to be understood well are as follows.

FIG. 1A: Diagnosis of FMF disease via a label-free plasmonic method with the invention. FIG. 1B: The plasmonic chip (3) was used in the invention. FIG. 1C: Separation of the plasmonic chip surface into two regions as control and sensor.

FIG. 2: Fabrication of the gold nanoparticle-based plasmonic chip (3).

FIG. 3A: Capturing the pyrin protein (17) on the plasmonic chip (3) surface. FIG. 3B: Change in the transmission response of the gold nanoparticles (14) covered with the anti-pyrin antibody (16) when tested with FMF-positive and FMF-negative samples. FIG. 3C: The change in the [1/integral] value calculated for the gold nanoparticles (14) covered with the anti-pyrin antibody (16) when tested with FMF-positive and FMF-negative samples. FIG. 3D: Creating the database using average and standard deviation values of the the calculated [1/integral] values for healthy and patient samples.

FIG. 4: The apparatus (22) used to divide the plasmonic chip (3) surface into two as control and sensor region.

### Definitions of Components/Pieces/Parts of the Invention

1. Light Source
2. Light Condensing Assembly
3. Plasmonic Chip
4. Stage
5. Fiber Focusing Assembly
6. Fiber
7. Spectrometer
8. Isolation Table
9. Case
10. USB Cable
11. Processing Unit
12. User Interface
13. Polymer Structure
16
14. Metal Nanoparticle
15. Glass Substrate
16. Anti-Pyrin Antibody
17. Pyrin Protein
18. Ligand Protein
19. Integral Area
20. Elastic Interlayer
21. Retaining Covers
22. Sample Preparation Apparatus
23. Pipetting Opening

### DETAILED DESCRIPTION OF THE EMBODIMENTS

The invention encompasses a portable biosensor developed for use in the diagnosis of FMF by determining the change in the pyrin protein produced in higher amounts in FMF patient samples compared to the pyrin found in healthy individuals using the high-precision sensing features of nanoparticle-based plasmonic chips.

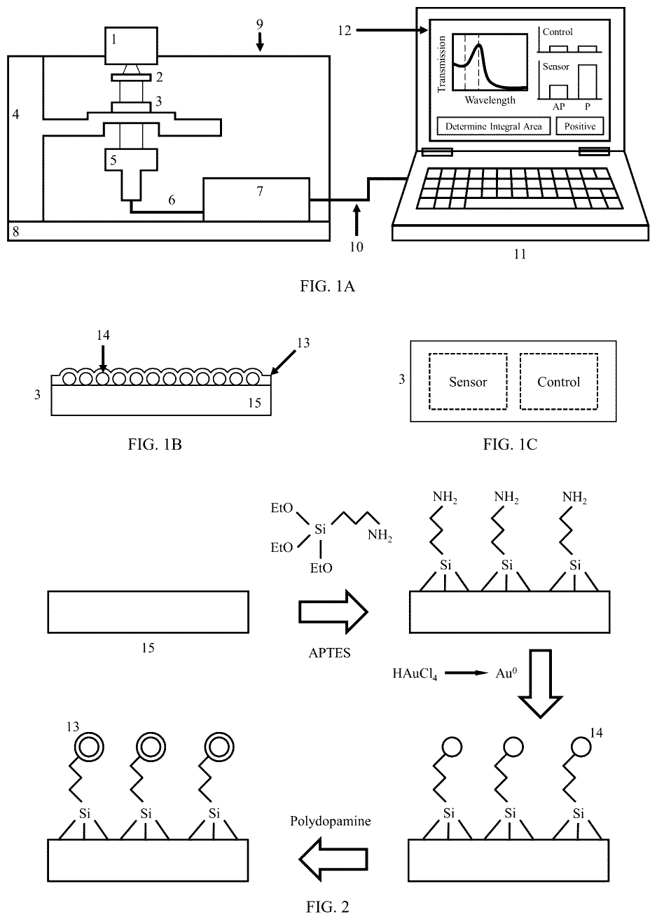

The main components of the invention include the nanoparticle-based plasmonic chip (3), the light source (1), and the spectrometer (7) (FIG. 1A). The plasmonic chips (FIG. 1B) on which the sensing tests will be performed in the invention are preferably based on nanoparticle synthesis on the surface of a substrate (15) made of glass without requiring cleanroom procedures (FIG. 2). For this reason, plasmonic chip fabrication is low-cost, and these plasmonic chips are disposable. The plasmonic chip (3) consists of metal nanoparticles (14) synthesized on the surface of a glass substrate (15) (FIG. 1B). Here, the metals that will form the nanoparticles are selected from noble metals (gold, silver, or platinum) due to their strong optical responses and resistance to oxidation and corrosion. In the synthesis of nanoparticles, in the case of the selection of metals (copper or aluminum) that have strong optical responses but can be oxidized, the surface of these nanoparticles after synthesis on the glass substrate surface is covered with a 2 nm glass (SiO₂) layer. Since the thickness of this layer is very thin, the interactions of the surface electromagnetic waves stimulated on the surface with the biomolecules are not affected. Thus, the corrosion of the nanoparticles is prevented without any negative effect on the biosensing capacity of the system.

The nanoparticles (14) are coated with a polymer structure (13). In the fabrication of the plasmonic chip (3), the surface of the glass substrate (15) is first cleaned with a piranha solution. (3-aminopropyl) triethoxy silane provides an amino functional property to the surface structure of the glass substrate (15). In the relevant reaction, the surface of the glass substrate (15) is reflexed (reversed) under acid catalyst and reflex temperature under left gel reaction conditions. Metal nanoparticle application is performed on the surface with a sufficient number of amino groups. In this application, a citrate reduction method is used. HAuCl₄ solution is added to the oil bath and 1% trisodium citrate solution. The reaction is continued at a strong mixing speed. After the process, the mixture is cooled at room temperature and a metal nanoparticle solution is obtained. Metal nanoparticles (14) obtained in appropriate size and morphology are coated on the surface of the glass substrate (15) under a nitrogen atmosphere and vacuum conditions by thermal annealing method. Unless the metal used is a noble metal, the glass surface is coated with 2 nm SiO₂ at this stage. The surface is then coated with polydopamine. These coating processes are carried out in the form of coating by dipping in 0.5 mg/mL polydopamine solution in the Tris buffer.

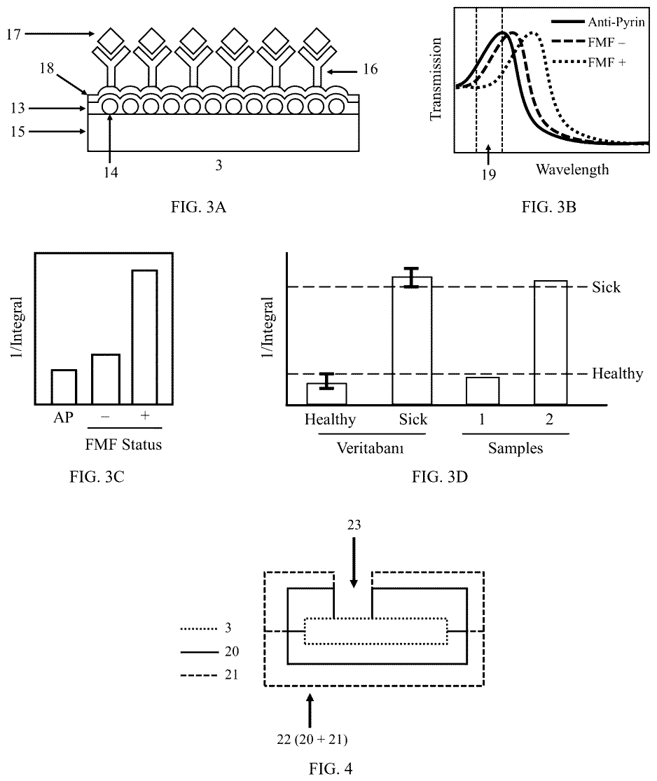

Metal nanoparticles (14) coated on the surface of the glass substrate (15) enable the detection of biological binding events by using the optical properties of special surface waves called plasmons (Stewart et al. 2008). The optical properties of metal nanoparticles (14) change when the refractive index in the vicinity of the metal surface changes, as plasmons are strongly localized on the surface of metals, and their excitations occurring at a specific wavelength depend on their size. These refractive index changes alter the excitation wavelength of the plasmons. Thus, the spectral variations in the optical responses of the nanoparticles (14) determines the presence of the bio-target to be identified in the tested sample. The optical method used by the invention is referred to as label-free sensing as it can identify bio-targets without the use of any optical label (e.g. fluorescence). The detection limits of the label-free sensing method of the present invention are solved by simultaneously monitoring the spectral change in more than one wavelength instead of a single spectral position. The spectral changes observed as a result of the binding of the analytes in the transmission response of the metal nanoparticles (14) used in the invention are calculated by integrating the transmission curve in a spectral window. Optical reading and spectral monitoring are also performed by monitoring the transmission response of nanoparticles as well as reflection, extinction, or absorption behaviors. This window, referred to the integral area (19), is defined with respect to the spectral position of the transmission response of the metal nanoparticles (14) at the time of the incubation of the anti-pyrin antibody (16) on the surface of the plasmonic chip (3). The spectral location where the transmission response is maximum and a spectral range containing smaller wavelengths from this location are selected. By selecting the endpoint of the integral area (19) as the maximum point corresponding to the binding of the anti-pyrin antibody (16), the effects of the Lorentzian shape of the transmission response of the nanoparticles (14) are eliminated in the integral calculation. The spectral integral is calculated and the spectral changes of the plasmonic mode excited by the metal nanoparticles (14) at different wavelengths are determined cumulatively. Thus, the sensitivity of the invention to the refractive index changes is enhanced compared to the principle relying on monitoring the spectral change in a single wavelength, and biosensing is ensured at low analyte concentrations. As seen in FIG. 3C, as a result of the binding of the pyrin protein (17) in the samples taken from healthy (FMF+) or sick (FMF−) individuals to the surface of the plasmonic chip (3), the transmission curve shifts to larger wavelengths and the [1/integral] value increases due to the decrease in the integral value. Since the amount of pyrin protein (17) in the patient samples is larger than the amount in the healthy samples, the [1/integral] value for the patient samples is higher than the healthy samples.

In case the plasmon excitation wavelength of the metal nanoparticles (14) differs in each plasmonic chip (3), the integral area (19) is automatically determined with the command given by the user in the user interface (12) before the test to be performed with each patient sample to determine the integral range. The user interface (12) determines the spectral location where the transmission response of the plasmonic chip (3) is maximum and the related integral area (19) before the binding of the pyrin protein (17). The program calculates the spectral integral value for the control and sensor regions (FIG. 1C) on the plasmonic chip (3) after the binding of the anti-pyrin antibody (AP) (16) and the pyrin protein (P) (17) (FIG. 1A).

FIG. 3B shows the transmission response of the metal nanoparticles (14) covered with the anti-pyrin antibody (16) for the two samples that are FMF negative and FMF positive. As a result of the incubation of these two samples on the plasmonic chip (3), it shows spectral shifts with different amounts based on the amount of pyrin protein in the samples. The concentration of pyrin protein, which is present in a certain amount in the healthy individuals, increases dramatically for FMF patients. Therefore, the amount of spectral shift in the transmission response of the nanoparticles (14) is much higher for the patient samples compared to the healthy samples (FIG. 3B). This large change in the amount of the spectral shift is analyzed by the invention for the diagnosis of FMF disease.

FIG. 3A shows the surface modification method used to capture the pyrin protein on the surface of the plasmonic chip (3). The analytes are attached to the surface of the plasmonic chip (3) by incubation and physical adsorption. The surface of the plasmonic chip (3) is washed with acetone, ethanol and deionized water and cleaned with UV-plasma. This cleaning protocol makes the surface of the plasmonic chip (3) suitable for analyte binding. First, a ligand protein (Example: A, G or A/G protein) (18) is coated on the surface of the plasmonic chip (3) by physical adsorption. The ligand protein (18) clings to the active groups in the polymer structure (13) on the surface of the metal nanoparticles (14). As shown in FIG. 3A, when the anti-pyrin antibody (16) is incubated on the surface of the plasmonic chip (3); since the ligand protein (18) shows a high affinity (attraction) to the Fc region of the anti-pyrin antibody (16), the antibodies are positioned to reveal their binding regions on the surface (like Y-shaped). In the last step, pyrin proteins (17) in the sample are attached to the surface thorough their capture by the binding sites of the anti-pyrin antibody (16) when incubated on the surface. In this protocol, unbound analytes are removed from the surface by rinsing the surface of the plasmonic chip (3) with deionized water and phosphate buffer solution (PBS) at the end of each incubation step.

A region on the plasmonic chip (3) is reserved for control (FIG. 1C) to address non-specific binding. The control region is used to eliminate the effects of chemical, physical or optical fluctuations on the FMF sensing signal for the samples under examination. Since there is no ligand protein in this region, the binding of another analyte other than the pyrin protein, which is the protein desired to be bound on the surface, is determined by the change in the transmission response of the gold nanoparticles (14). The spectral variations detected in the control region eliminate the effects of non-specific bindings on the sensing test. Minimal or zero spectral integral change in the control region indicates the reliability of the test results. The invention has a sample preparation apparatus (22) for defining two regions on the plasmonic chip surface called as control and sensor. The plasmonic chip (3) is placed between the two elastic interlayers (20) in this apparatus. There is a pipetting opening (23) in the upper elastic layer where bio-agents can be pipetted and incubated on the surface of the plasmonic chip (3). In the lower elastic layer, there is a hole with the size of the plasmonic chip (3). This hole and the plasmonic chip (3) are automatically aligned with the pipetting opening (23) in the sample preparation apparatus (22). The elastic interlayers (20) are fixed with two retaining covers (21). By compressing the elastic interlayers (20) of the retaining covers (21), the surface of the plasmonic chip (3) is sealed and the bio-agents can be incubated only in the desired region (sensor area). Since the surface of the control region is covered with the upper layer, this area is not exposed to any bio-agent. While sealing is ensured with the elasticity of the interlayers, the plasmonic chip (3) is not broken thanks to these layers, which are deformed by the compression using the covers.

The components in the preferred embodiment of the invention are (FIG. 1A): a plasmonic chip (3) based on the metal nanoparticle (14), a light source (1), a fiber focusing assembly (5), a spectrometer (7) and the case (9) in which the stage (4) is placed on an isolation table (8), a user interface (12) in which the spectral changes in the optical response of the plasmonic chip are calculated and examined. As seen in FIG. 1A, a broadband light source (1) (Example: white halogen or LED) is positioned perpendicular to the plasmonic chip (3) and the light illumination on the surface of the plasmonic chip (3) is ensured to be homogeneous with a light condensing assembly (2). The light passing through the plasmonic chip (3) is collected with a fiber focusing assembly (5) (Example: consisting of an achromatic lens, an objective lens, and a fiber collimator), transferred to the fiber (6) and transmitted to the spectrometer (7). The spectrometer 21 used in the system makes measurements with a high optical accuracy and it is portable. In addition, a stage (4) that provides movement in the x, y and z directions is used to obtain data from a region of the plasmonic chip (3) determined by the user. These components are positioned in a case (9) to eliminate the noise that may be caused by the ambient light. The entire optical system is placed on a isolation table (8) to prevent vibrations that may be caused by external factors and thus the system can be used even in the outdoor settings without the need for a laboratory environment. The spectrometer (7) is connected to the processing unit (11) with a USB cable (10), which is preferably a portable computer (a laptop). The spectral data generated by the spectrometer is transferred to a user interface (12).

There is a database (FIG. 3D) created by determining the mean spectral integral change and standard deviation values for the samples collected from multiple patients and healthy individuals and this database is integrated into the user interface (12). The transmission response of the metal nanoparticles (14) in the sensor region is measured by the spectrometer (7) before and after the incubation of the examined sample on the plasmonic chip (3). Comparative analysis is conducted based on the corresponding range in the database for the amount of spectral change in the transmission response.

The use of the invention begins with the blood drawn from patients who visit a health institution and show FMF symptoms. Serum-containing pyrin protein is obtained from the patient's blood sample with classical laboratory methods. The samples to be tested with the invention are in the form of serum; in this way, a reliable and selective identification signal is produced by preventing non-specific bindings that may occur due to a sample, e.g., blood, containing many bio-materials. The serum is incubated on the plasmonic chip surface in the system.

The chip is placed in the biosensor after incubation. The pyrin protein (17) in the serum is captured on the surface of the metal nanoparticle (14) in the plasmonic chip, and the amount of the variation in the transmission response of the metal nanoparticles (14) created by the captured pyrin protein (17) is compared with the spectral variation amounts measured in the patient and healthy individuals with the user interface in the system and these measurements are provided to the operator.

What is claimed is:

1. A diagnostic method for (Familial Mediterranean Fever (FMF) disease, comprising the following process steps:
determining a change value in a transmission response and/or reflection, extinction or absorption behaviors of nanoparticles depending on an amount of a pyrin protein in blood derivatives, and
comparing the change value with a determined reference value.

2. A biosensor system for use in the diagnostic method according to claim 1, comprising;
a substrate acting as a surface with an amino functional feature incorporated into a surface structure of the substrate,
a metal nanoparticle coated on the substrate and coated with a polymer structure,
a ligand protein clung to the polymer structure on a surface of the metal nanoparticles,
a plasmonic chip containing an anti-pyrin antibody positioned on a surface of the ligand protein wherein the pyrin protein binds to the anti-pyrin antibody,
at least one light source,
at least one spectrometer detecting a light signal reflected or absorbed from the a surface of the plasmonic chip, and
a processing unit wherein the change value in the transmission response of the metal nanoparticles in the plasmonic chip is compared with the determined reference value in the processing unit.

3. The biosensor system according to claim 2, further comprising a sample preparation apparatus configured for dividing the surface of the plasmonic chip into a control region and a sensor region.

4. The biosensor system according to claim 2, further comprising a light condensing assembly for homogeneous light illumination on the surface of the plasmonic chip.

5. The biosensor system according to claim 2, further comprising a stage providing movement in x, y, and z directions to obtain data from a determined region of the plasmonic chip.

6. The biosensor system according to claim 2, further comprising a fiber allowing a light to be transmitted through the plasmonic chip to the at least one spectrometer.

7. The biosensor system according to claim 2, further comprising a fiber focusing assembly for collecting a light passing through the plasmonic chip and transferring the light to a fiber.

8. The biosensor system according to claim 2, further comprising a portable isolation table preventing vibrations, wherein the vibrations arise from external factors.

9. The biosensor system according to claim 2, further comprising a case, wherein an isolation table is placed in the case, and the case eliminates noise.

10. The biosensor system according to claim 2, further comprising a USB cable for connecting the at least one spectrometer to the processing unit.

11. The biosensor system according to claim 2, further comprising a user interface, wherein a comparison is displayed to a user.

12. The biosensor system according to claim 2, wherein the processing unit is a laptop.
